(19) United States
(12) Patent Application Publication
(10) Pub. No.: US 2016/0346412 A1
(43) Pub. Date: Dec. 1, 2016

(54) A KIT FOR PREPARING A RADIOPHARMACEUTICAL

(71) Applicant: THE SOUTH AFRICAN NUCLEAR ENERGY CORPORATION LIMITED, Brits Magisterial District (ZA)

(72) Inventors: Jan Rijn Zeevaart, Florauna (ZA); Zoltan Szucs, Debreen (HU); Judith Wagener, Mogale City (ZA)

(21) Appl. No.: 15/117,167

(22) PCT Filed: Feb. 6, 2015
(86) PCT No.: PCT/IB2015/050915

§ 371 Date: Aug. 5, 2016

(30) Foreign Application Priority Data
Feb. 7, 2014 (GB) 1402132.3

(51) Int. Cl. A61K 51/12; C07B 59/00; A61K 51/04

(52) U.S. Cl. CPC A61K 51/1282; A61K 51/0491; C07B 59/005; C07B 2200/05

(57) ABSTRACT

The present invention relates to a stabilized kit for the preparation of a radiopharmaceutical. In particular, the present invention relates to the use of a non-aqueous solvent for the stabilisation of the ligand component of the kit.

### BACKGROUND OF THE INVENTION

This invention relates to a stabilized kit for preparing a radiopharmaceutical. In particular, this invention relates to the use of a non-aqueous solvent for the stabilization of the ligand component of the kit.

Radiopharmaceuticals have to be prepared and administered within a limited time due to short half-life of most radionuclides used in applications. It is usually formulated from kits produced under GMP conditions. A kit generally contains the applicable ligand to which the radionuclide, such as ^{99m}Tc, is to be complexed, an adequate quantity of reducing agent, buffer to adjust the pH to suit the optimum labelling conditions, stabilizing agents and excipients. The kits are prepared in a lyophilized or freeze-dried form that increases the stability and shelf life. The kits can easily be transported and stored before reconstituted using the indicated radionuclide. The freeze dried kits simplify labeling and ensure more stable conditions for labeling.

The availability of a freeze dried kit formulation is advantageous for hospital personnel responsible for easily preparing the radiopharmaceutical for administration since it only involves the addition of the radionuclide and heating if required. These preparation steps are therefore within the ability of the responsible person at the hospital.

An example of a radiopharmaceutical is ^{99m}Technetium-ethylenedicysteine deoxyglucosamine (^{99m}Tc-ECDG) 1. ^{99m}Tc-ECDG is a single photon emission computed tomography (SPECT)/computed tomography (CT) (SPECT/CT) imaging agent that is currently in phase three clinical trials in the USA for its ability to detect primary lesions of lung cancer¹. The imaging capabilities of ^{99m}Tc-ECDG are comparable to ¹⁸F-fluorodeoxyglucose (¹⁸F-FDG) 2, a positron emission tomography (PET)/CT imaging agent, which is extensively utilized (more than 95% of scans) for the detection of hibernating myocardium and metabolically active cancer tissue². The major driving force behind the potential implementation of ^{99m}Tc-ECDG over ¹⁸F-FDG is the significantly lower costs associated with employing a SPECT radiotracer compared to a PET radiotracer and achieving the same level of quality and efficiency in lung cancer imaging³.

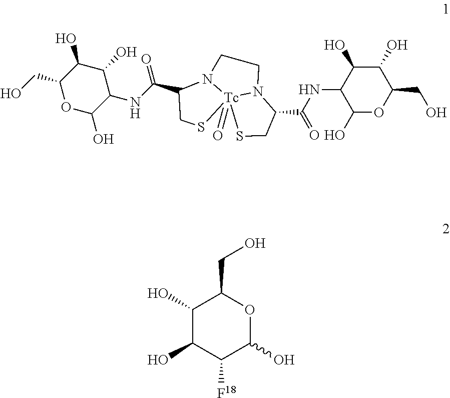

The mechanism of action of ^{99m}Tc-ECDG is proposed to occur via the hexosamine pathway, as a result of containing two glucosamine substituents. Glucosamine enters cells through the hexosamine biosynthetic route and its regulatory products of glucosamine-6-phosphate mediate insulin activation downstream and signal glycosylation and cancer growth². In the hexosamine pathway, up-regulated glucose transporters promote the overexpression of glutamine: fructose-6-phosphate amidotransferase (GFAT). Phosphorylated glucosamine binds to uridine diphosphate (UDP) to form UDPN-acetylglucosamine (UDP-GLcNAc). The glycosylation of serine and threonine residues on nuclear and cytosolic proteins by O-linked protein N-acetylglucosamine (O-GlcNAc) transferase is common in all multicellular eukaryotes. Glycosylation is a part of posttranslational modification and seems to modify a large number of nucleocytoplasmic proteins. O-GlcNAc transferase activity is highly receptive to intracellular UDP-GLcNAc and UDP concentrations, which are in turn highly sensitive to glucose concentrations and other stimuli. Within the cell nucleus, the ubiquitous transcription factor Sp1 is highly modified by O-GlcNAc. Sp1 undergoes hyperglycosylation in response to hyperglycemia or elevated glucosamine. Since O-GlcNAc is involved in the hexosamine pathway and nucleus activity, it becomes an appealing imaging agent for differential diagnosis in tumours.

A survey of the literature on the published syntheses, (references [5], [6], [7] and [8]), gives an overview of a few experimental methods on how to produce ECDG 3. Unfortunately none of these published procedures were successfully reproducible as these syntheses involve exposing ECDG to an aqueous medium which proved futile as ECDG has been shown to be air, light, water and temperature sensitive⁹. The synthesis of ECDG has been described to be a challenging task, given the highly labile nature of this ligand⁹. Since ECDG is intended for use as an imaging agent, the material has to be of pharmaceutical grade which means that purification steps will need to be undertaken without a substantial loss in yield. This will prove to be highly difficult because of the low stability of ECDG.

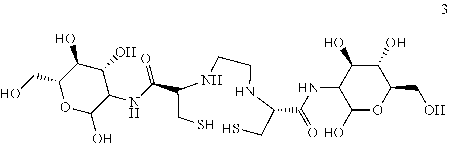

A second factor compounding to the problem of making ^{99m}Tc ECDG useful as a radiopharmaceutical in the nuclear medicine setting is its presentation in a kit formulation.

The production of kits of ECDG, a water labile ligand, is problematic as the normal kit procedure includes a lyophilisation step in the aqueous phase wherein the pure ligand active pharmaceutical ingredient (API) is dissolved in water/saline containing at least one each of a reducing agent, additive and buffer, distributed in vials and freeze dried. At the hospital the ^{99m}Tc in saline is added to the kit and reconstituted. The ^{99m}Tc is then chelated to the ECDG ligand and the ^{99m}Tc-ECDG radiopharmaceutical is ready for injection. The inventors have found that ECDG breaks down in water almost immediately. Only when a metal ion is chelated to the ECDG, such as in the case of ^{99m}Tc-ECDG, is it stable in water.

A need therefore exists for a kit system that includes stabile components, which allows for a simple, repeatable and stable labeling technique, suitable for diagnostic, therapeutic or other tracer applications. Further, there exists a need for the effective radiolabelling of ligands, at radiochemical purity levels which are acceptable for regulatory approval and whilst maintaining high stability, purity and yield.

### SUMMARY OF THE INVENTION

According to a first aspect of the invention, there is provided a kit for preparing a radiopharmaceutical, the kit comprising:
a) a ligand dissolved in a non-aqueous solvent, the ligand being capable of bonding to a radionuclide and wherein the solvent is selected from the relative polarity range of hexane to glycerine;
b) a reducing agent;
c) a buffer solution;
d) and optionally additives such as weak chelating agent, anti-oxidant, solubiliser or bulking agent
   and wherein components a), b), c) and d) are each in a lyophilized form.

In a preferred embodiment of the invention, the reducing agent is a mixture of SnCl₂ or SnF₂ or stannous tartrate, hydrochloric acid and water, and the buffer solution is a phosphate or citric acid or acetate buffer solution. Alternatively, the buffer is a combination of any one of a phosphate, citric acid or acetate buffer solution.

Preferably, the weak chelating agent is selected from DTPA, glucoheptonate, tartrate and medronate, or a combination of any. The anti-oxidant is selected from gentisic acid, ascorbic acid and para amino benzoic acid, or a combination thereof. The solubiliser is selected from gelatin or cyclodextrin, or a combination thereof and the bulking agent is selected from mannitol, inositol, glucose and lactose, or a combination thereof.

The components a), b), c) and d) may be contained in one vial. Alternatively, components b), c) and d) are contained in a first vial and component a) is contained in a second vial.

The ligand may be selected from ECD, HMPAO, MAG3, and MIBI or alkali metal salts thereof, or alkaline earth metals thereof. Preferably, the ligand is ECDG or an alkali metal salt thereof. The solvent is selected from: methanol, ethanol, ethyl acetate, hexane, chloroform, dichloromethane, toluene, ether, tetrahydrofuran and acetonitrile, or a combination thereof. Preferably, the solvent is selected from methanol or ethanol. More preferably, the solvent is methanol.

The metal radionuclide may be selected from ^{99m}Tc, ¹⁸⁸Re, ¹⁸⁶Re, ¹⁵³Sm, ¹⁶⁶Ho, ⁹⁰Sr, ⁹⁰Y, ⁸⁹Sr, ⁶⁷Ga, ⁶⁸Ga, ¹¹¹In, ¹⁵³Gd, ⁵⁹Fe, ⁵²Fe, ²²⁵Ac, ²¹²Bi, ⁴⁵Ti, ⁶⁰Cu, ⁶¹Cu, ⁶²Cu, ⁶⁴Cu, ⁶⁷Cu, ^{195m}Pt, ^{191m}Pt, ^{193m}Pt, ^{117m}Sn, ¹⁰³Pd, ^{103m}Rh, ⁸⁹Zr, ¹⁷¹Lu, ¹⁶⁹Er, ⁴⁴Sc, ¹⁵⁵Tb, ¹⁴⁰Nd, ¹⁴⁰Pr, ¹⁹⁸Au, ¹⁰³Ru, ¹³¹Cs, ²²³Ra, ²²⁴Ra and ⁶²Zn.

Preferably the radionuclide is ^{99m}Tc, ¹⁰³Pd, ^{103m}Rh, ^{195m}Pt, ^{193m}Pt, ¹⁹¹Pt. More preferably, the radionuclide is ^{99m}Tc.

The kit further comprising instructions for use.

### BRIEF DESCRIPTION OF THE DRAWINGS

FIG. 1 is a mass spectrum of the ECDG produced

### DESCRIPTION OF PREFERRED EMBODIMENTS

The kits were prepared according to the following.

The following solutions were prepared under Ar(g) conditions to ensure the absence of CO₂ or O₂:
a) An adequate amount of ECDG or salt thereof is dissolved in a non-aqueous solvent, in the relative polarity range of hexane to glycerin.
b) Phosphate/citric acid buffer solutions at the appropriate pH for optimum labeling conditions
c) Stannous salt solution in a neutral or acidic medium, which acts as a reducing agent of the pertechnetate ion (^{99m}TcO₄⁻) in oxidation state VII to IV to ensure ^{99m}Tc is chemically reactive to bind with the ligand, ECDG.

For a two vial kit formulation the freeze drying procedure, using solutions described above, involves the following:
a) Vial 1: A sufficient volume of the ECDG solution was added to Vial 1, frozen and then freeze dried under Ar(g) conditions.
b) Vial 2: A predetermined volume of the prepared phosphate/citric acid buffer solution was added to the Ar(g) filled Vial 2, frozen and freeze dried overnight followed by adding the Sn solution (60-100 μg Sn(II)), followed by freeze drying under Ar(g) conditions.

All the vials are stored in dark conditions in the freezer. The labeling protocol entails the reconstitution or dissolution of Vial 1, the addition of Vial 1 to Vial 2 immediately followed by the addition of an adequate ^{99m}Tc activity. The reaction mixture is heated (60-80° C.) for a limited time to ensure labeling. Quality Control with TLC and HPLC should record >90% labeling and radiochemical purity of more than 95%.

For a one vial kit formulation the freeze drying procedure, using solutions described above, involves the following:
a) Firstly a predetermined volume of the prepared phosphate/citric acid buffer is frozen and freeze dried. Then a Sn solution (60-100 μg Sn(II)) was added to the Ar(g) filled vial and frozen, followed by freeze drying under Ar(g) conditions.
b) Lastly the pure ECDG is dissolved in a non-aqueous solvent on top of the freeze dried material of a), frozen and freeze dried. This labeling protocol entails reconstitution only by the addition of an adequate ^{99m}Tc activity. The reaction mixture is heated (60-80° C.) for a limited time to ensure labeling. Quality Control with TLC and HPLC should record >90% labeling and radiochemical purity of more than 95%. added to the kit and constituted ready for injection.

In the preparation of the kits, the ECDG was synthetically prepared by the Applicant. A synthetic route to produce ECDG was successfully carried out in five synthetic steps, starting from commercially available L-thiazolidine-4-carboxylic acid. The synthesis route can be briefly summarized as follows.

^{99m}Tc-ECDG from a structural perspective can be considered to consist of three components, that is: (i) an L, L-ethylene dicysteine (EC) ligand at its core, (ii) two cancer targeting D-glucosamine groups and (iii) a ^{99m}Tc radionuclide. EC can be obtained from the radical promoted dimerization reaction of the commercially available L-4-thiazolidinecarboxylic acid [10]. The thiol and secondary amine functionalities of EC are reactive sites and have been shown to be effectively and efficiently masked by benzyl (Bn) [11] and benzyl chloroformate (Cbz) protecting groups respectively. The two D-glucosamine groups can be theoretically coupled to the acid moieties of EC via a mixed anhydride coupling reaction by employing the reagent ethyl chloroformate. ECDG can then be afforded by the global deprotection of the coupling reaction product in a sodium/ammonia solution [8]. This reaction can be quenched with ammonium phenylacetate which would produce a 2-propanol soluble sodium phenylacetate salt that would allow for adequate purification of the ECDG from reaction by-products. This synthesized ECDG can then by labeled with ^{99m}Tc and utilized as need be.

The synthesis of EC 4

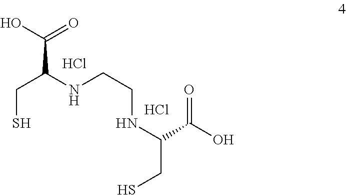

from L-thiazolidine-4-carboxylic acid was carried out exactly as the literature stipulated [10] and afforded the desired product in a 38% yield. Once the reaction had gone to completion, the ammonia rapidly evaporates (boiling point is −33° C.) and the resultant residue is dissolved in water to give a highly basic (pH=12.0) solution. Thus, 5M HCl is added to protonate the basified EC ligand and precipitate the molecule as its dihydrochloride salt, which is achieved at pH 3.0-2.0. The starting material, L-thiazolidine-4-carboxylic acid, is soluble in acidic media and remains in solution and therefore this step serves as the first stage of EC 4 purification. The precipitated EC 4 is then filtered and it was discovered that the immediate recrystallization of this crude EC 4 from boiling ethanol, followed by drying of the material under high vacuum, yielded pure EC 4 as a powdery white solid. The NMR of EC 4 was carried out in D₂O, with the necessary addition of 6.0 equivalents of K₂CO₃ to (i) neutralise the dihydrochloride salt and (ii) deprotonate the thiol and acid functionalities, which allowed for EC 4 to be solubilised and analysed. The proton and carbon NMR data of EC 4 was in accurate accordance with the literature data, along with the determined melting point. This data also depicted that the purity of the EC 4 was greater than 99%.

EC 4 was benzylated according to the reference information [10] and no deviations from this were observed. This protection step was necessary as the thiol groups would also react in the planned glucosamine coupling reaction and therefore required to be masked. There is however no literature available on the proton or carbon NMR data of EC-Bn 5

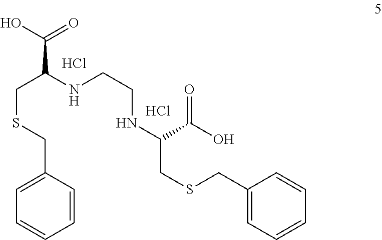

and thus a solvent system and method of analysis had to be determined. It was experimentally found that EC-Bn 5 fully dissolved in a mixture of D₂O and deuterated DMF in a 6:4 v/v ratio along with the addition of 4.0 equivalents of K₂CO₃, which served to neutralise the dihydrochloride salt of EC-Bn 5 and deprotonate the two acid moieties. This allowed for the NMR data of EC-Bn 5 to be generated and serves as the first reported proton and carbon NMR spectra on this compound. The proton spectrum closely resembles that of the parent EC 4 compound but contains the benzyl CH₂ protons as a singlet at 4.69 ppm and the ten aromatic protons appearing at 7.16 ppm as a multiplet. The carbon NMR spectrum correlates with findings of the proton NMR spectrum as the CH₂ carbon atoms are observed at 35.9 ppm and the signals at 127.1 ppm, 128.6 ppm, 128.8 ppm and 138.6 arise from the aromatic ring. This data, along with the determined melting point that fits within the expected literature range, confirms that the benzyl protection was successfully achieved.

The secondary amine moieties of EC-Bn 5 were protected with benzyl chloroformate protecting groups. Similarly to the thiol groups, these secondary amine groups would also react in the planned glucosamine coupling reaction and therefore also required to be capped. The EC-Bn Cbz protection was initially carried out for 2 h at 0° C. and then for 16 h at room temperature (RT). A diethyl ether washing step was required to remove any unreacted CbzCl, followed by acidification of the aqueous medium to pH 3.0 to protonate the carboxylic acid group of EC-Bn-Cbz 6

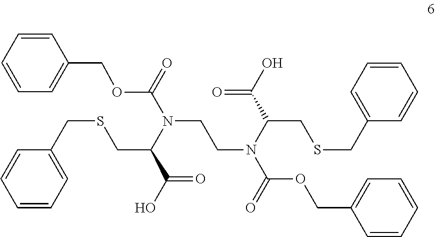

which resulted in the precipitation of the product as a white solid. It was found that the product dissolved in large volumes of organic solvent and the extraction of the acidified solution with ethyl acetate allowed for EC-Bn-Cbz 6 to be isolated. The separation and subsequent solvent removal of the organic phase yielded the desired product as an amorphous solid. The EC-Bn-Cbz 6 had to be dried thoroughly in the presence of a high vacuum to ensure that the material was completely free of traces of solvent or water. The EC-Bn-Cbz 6 product rapidly decomposed on silica gel and thus could not be purified further, which was found to be contrary to the published data [12]. A solvent system for the NMR analysis of EC-Bn-Cbz 6 could not be determined and this was also not in accordance with the literature information that gives the NMR data in CDCl₃. The LC-MS analysis of this product also proved unsuccessful as a result of the benzyl protecting groups which are notoriously problematic for MS determination. Thus the crude EC-Bn-Cbz 6 was used directly into the next step.

The coupling reaction of EC-Bn-Cbz 6 and tetra-acetylglucosamine was carried out employing ethyl chloroformate as the coupling reagent. The reaction conditions and work up were performed in the same manner as those found in the prior art, but a new column purification solvent system was determined. It was found that a three component solvent combination of methanol (MeOH), ethyl acetate (EtOAc) and hexane in a ratio within the range of (1-5):(10-90):(10-80) allowed for the fully protected ECDG 7 to be isolated at a higher purity.

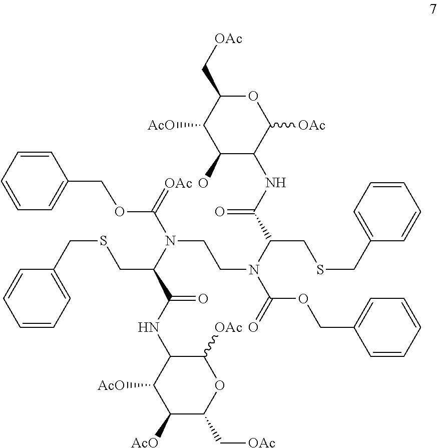

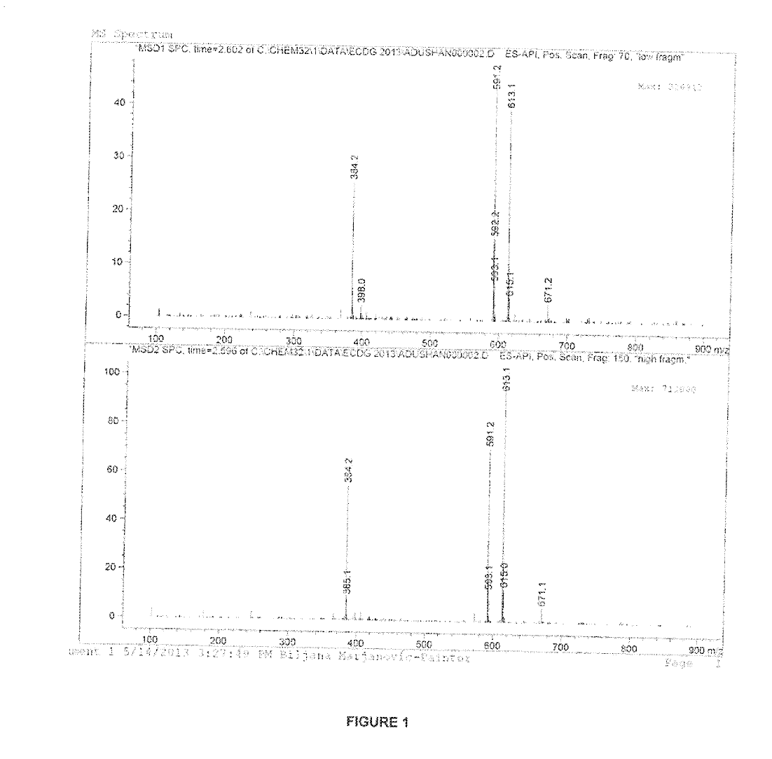

The last step was the sodium/ammonia facilitated global deprotection of fully protected ECDG 7 to yield ECDG 3. The fully protected ECDG 7 was reacted with 20.0 equivalents of sodium metal to completely remove the acetate, Cbz and Bn protecting groups. The reaction was then quenched with the addition of 12.0 equivalents of ammonium phenyl acetate which resulted in the formation of sodium phenyl acetate as a by-product. The sodium phenyl acetate was removed from the reaction mixture, once the ammonia liquid was evaporated under an argon gas atmosphere, by a 2-propanol washing step. Sodium phenyl acetate is highly soluble in 2-propanol whilst ECDG 3 is not, and thus the organic medium is filtered under an inert atmosphere to afford ECDG 3 as a cream coloured, strong-smelling solid. This ECDG 3 was then washed with diethyl ether and then dried under a high vacuum for 1 h with the exclusion of light. The identification and purity of this ECDG was determined by MS (FIG. 1) and the required MS-peak for ECDG 3 was observed at 591.1 units. The ECDG 3 was stored under argon, in the absence of light at −20° C.

### EXAMPLES

### Example 1

### Double Vial Kit

### Lyophilization Protocol

a) To a solution of sodium phosphate dibasic (0.284 g, 0.002 mol) in water (de-oxynated) citric acid (0.201 g, 0.001 mol) was added to result in a pH 5.5 Phosphate/citric acid buffer solution. 855 μl prepared phosphate/citric acid buffer solution was added to the first Argon filled vial, closed and frozen before freeze drying overnight.
b) Hydrochloric acid (0.10 ml, 0.1 M) was added to tin(II) chloride dihydrate solution (0.01 g, 0.04 mmol) and diluted to 10 ml with water (de-oxynated). 100 μl Sn solution (=60 μg Sn(II)) was then added to vial 1, frozen followed by freeze drying.
c) Methanol (1.5 ml) was added to a second argon filled vial with ECDG (10 mg, 0.017 mmol). The vial was immersed into liquid nitrogen to freeze the solvent and freeze dried. The vial should be kept in the dark and freezer.

Note that all vials should be filled with Ar to ensure the absence of CO₂ or O₂.

### Labelling Protocol

a) Add 355 μl H₂O to freeze dried ECDG vial.
b) Transfer to freeze dried Buffer/Sn vial and add a small magnetic stirrer bar. Vortex to dissolve buffer salts.
c) Immediately followed by the addition of 500 μl TcO₄⁻ (or equivalent volume for activity of approx. 40 mCi).
d) Place on hotplate and stir for 15 min at 70° C.
e) TLC and HPLC-QC is performed.

### Example 2

### One Vial Kit

### Lyophilization Protocol

a) To a solution of sodium phosphate dibasic (0.284 g, 0.002 mol) in water (de-oxynated) citric acid (0.201 g, 0.001 mol) was added to result in a pH 5.5 Phosphate/citric acid buffer solution. 855 μl prepared phosphate/citric acid buffer solution was added to the first Argon filled vial, closed and frozen before freeze drying overnight.
b) Hydrochloric acid (0.10 ml, 0.1 M) was added to tin(II) chloride dihydrate solution (0.01 g, 0.04 mmol) and diluted to 10 ml with water (de-oxynated). 100 μl Sn solution (=60 μg Sn(II)) was then added to vial 1, frozen followed by freeze drying.
c) Methanol (1.5 ml) was added to a second argon filled vial with ECDG (10 mg, 0.017 mmol). This was quantitatively transferred to vial 1 containing the Sn/Buffer. Immerse the vial into liquid nitrogen to freeze the solvent and freeze dried. The vial should be kept in the dark and freezer.

Note that all vials should be filled with Ar to ensure the absence of CO₂ or O₂.

### Labelling Protocol

a) Add 500 μl TcO₄⁻ (or equivalent volume for activity of approx. 40 mCi) to the vial containing ECDG, Sn and buffer.
b) Place on hotplate and stir for 15 min at 70° C.
c) TLC and HPLC-QC is performed.

### Example 3

### Synthesis of ECDG

### The synthesis of L, L-Ethylenedicysteine.2HCl

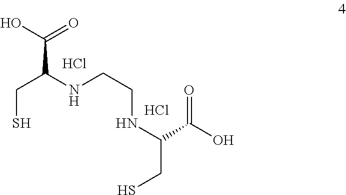

L-thiazolidine-4-carboxylic acid (30.0 g, 225 mmol) was slowly added to liquid ammonia (150 ml) in a two-necked round bottom flask, equipped with cooling condenser (filled with liquid nitrogen), argon gas inlet and an oil-filled outlet trap. The mixture was vigorously stirred till all the L-thiazolidine-4-carboxylic acid had completely dissolved followed by adding cleaned sodium metal (8.00 g, 349 mmol, 1.50 equivalents) portion-wise over 15 minutes. Once addition of the sodium metal was complete, a deep-blue colour was observed, and this solution was stirred for 20 minutes at room temperature. Ammonium chloride was then carefully added in spatula-tip portions, until the mixture became a white colour and all the unreacted sodium metal had been quenched. The ammonia solvent was then allowed to evaporate and the resulting reaction residue was dissolved in water (200 ml) and the pH was adjusted to 3.0 with concentrated HCl, which resulted in the precipitation of the dihydrochloride salt of ethylenedicysteine as a white solid. The product was collected by vacuum filtration, recrystallised from boiling ethanol and dried under high vacuum to afford 14.7 g (38%) of ethylenedicysteine.2HCl 4

M.p.: 252-254° C. (reference 251-253° C.[10]);

¹H NMR (400 MHz, D₂O and 6.0 equivalents of K₂CO₃):

δ_{H}=3.27 (2H, t, 2×CH—COOH), 2.70-3.00 (8H, m, 2×CH₂—N and 2×CH₂—SH overlapped), 2.62 (2H, m, 2×NH)².

¹³C NMR (400 MHz, D₂O and 6.0 equivalents of K₂CO₃):

δ_{C}=177.9 (COOH), 65.6 (CH—N), 44.8 (CH₂—N), 26.8 (CH₂—SH).

### Synthesis of S,S′-dibenzyl ethylenedicysteine.2HCl 5

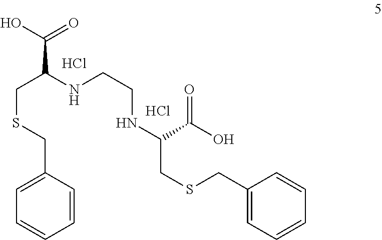

Ethylenedicysteine.2HCl 4 (2.0 g, 6.0 mmol) was dissolved in 2M NaOH (30 ml) at room temperature and ethanol (40 ml) was added, and the resulting solution was stirred vigorously for 20 min. Benzyl chloride (1.48 g, 11.7 mmol, 2.0 equivalents) in dioxane (20 ml) was added dropwise to the ethylenedicysteine solution and then stirred for a further 30 min after the addition was complete. The ethanol and dioxane were then removed in vacuo and then pH of the resulting aqueous mixture was acidified to pH 3.0 with 5M HCl. This resulted in the precipitation of the hydrochloride salt of S,S′-dibenzyl ethylenedicysteine 5 which was filtered under vacuum and dried under high vacuum in a 85% (2.7 g) yield.

M.p.: 227-228° C. (reference 251-253° C.);

¹H NMR (400 MHz, D₂O/DMF (6:4 v/v ratio) and 4.0 equivalents of K₂CO₃):

δ_{H}=7.16 (10H, m, 2×CH₂—C₆H₅), 3.68 (4H, s, 2×CH₂—C₆H₅) 3.14 (2H, t, CH—COOH), 2.44-2.85 (10H, m, 2×CH₂—N, 2×CH₂—SH and 2×NH overlapped);

¹³C NMR (400 MHz, D₂O/DMF (6:4 v/v ratio) and 4.0 equivalents of K₂CO₃):

δ_{C}=179.5 (COOH), 138.6 (Ar—C), 128.0 (Ar—C), 128.6 (Ar—C), 127.1 (Ar—C), 62.7 (CH—N), 46.6 (CH₂—N), 35.9 (CH₂—C₆H₅), 34.4 (CH₂—SH).

### Synthesis of Fully-Protected Ethylenedicysteine Deoxyglucosamine 7

S,S′-dibenzyl ethylene dicysteine 5 (6.0 g, 11.5 mmol) was dissolved in 10% K₂CO₃ solution (150 ml) and cooled to 0° C. in an ice bath. A mixture of benzyl chloroformate in dioxane (150 ml) was then quickly added to the solution which then stirred for 2 hours at 0° C. The cooling bath was then removed and the mixture was stirred for 16 h at RT, before being extracted with diethyl ether (2×50 ml). The aqueous layer was then carefully acidified to pH 3.0 with 1 M HCl which resulted in the precipitation of a white compound. Ethyl acetate (200 ml) was added and the precipitated solid dissolved into this organic layer with vigorous stirring. The organic layer was separated, dried over anhydrous magnesium sulphate, filtered and the solvent was removed on a rotary evaporator. The resulting clear residue was then dried on a high vacuum to afford 5.75 g (70% yield) of crude N, N′-dibenzyloxycarbonyl-S,S′-dibenzyl ethylenedicysteine 6 as an amorphous solid. This compound was unstable to purification and insoluble in the tested NMR solvents and was consequently used directly into the next reaction.

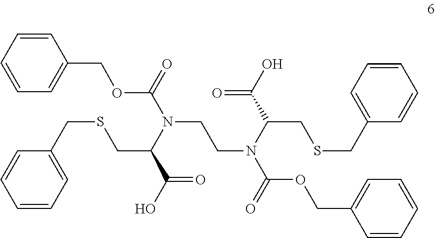

EC-Bn-CBz 6 (1.34 g, 1.87 mmol) was dissolved in dry chloroform (30 ml) with triethylamine (0.378 g, 3.74 mmol, 2.0 equivalents) and the solution was cooled to −15° C. in a sodium chloride/ice slurry cooling bath under an argon atmosphere. Ethyl chloroformate (0.406 g, 3.74 mmol, 2.0 equivalents) was added dropwise and the resulting mixture was stirred for a further 15 min. To this reaction mixture, a solution of tetra-acetylglucosamine (1.58 g, 4.11 mmol, 2.2 equivalents) and triethylamine (0.416 g, 4.11 mmol, 2.0 equivalents) in dry chloroform (30 ml) was added, and the combined reaction mixture was stirred for 1 h at 0° C. and then 12 h at RT. The solution was then successively washed with 1 M HCl (2×25 ml), a 5% K₂CO₃ solution (2×25 ml), H₂O (50 ml), dried over anhydrous magnesium sulphate, filtered and the solvent was removed in vacuo. The residue was purified by column chromatography (silica gel 60; mobile phase: MeOH/EtOAc/Hexane) to afford 1.80 (70% yield) g of fully-protected ethylenedicysteine deoxyglucosamine 7 as a white crystalline solid.

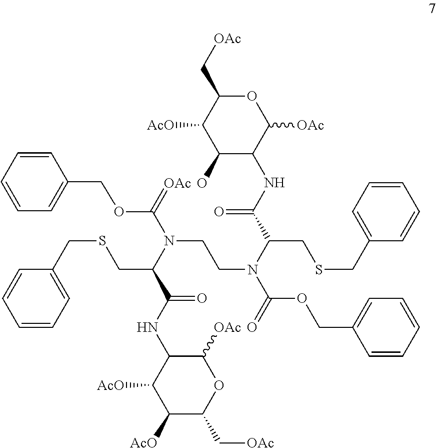

¹H NMR (400 MHz, CDCl₃):

δ_{H}=8.62 (2H, s, 2×NH), 7.48-7.40 (20H, m, 2×OCH₂—C₆H₅, 2×SCH₂—C₆H₅), 6.04 (2H, d, tetrahydropyrananomeric proton), 5.45-5.20 (6H, m, 2×OCH₂—C₆H₅, 2×tetrahydropyran protons overlapped), 4.48-4.07 (6H, m, 2×CH—CONH, 4×tetrahydropyran protons overlapped), 3.72-3.48 (12H, 4×tetrahydropyran proton, 4×CH₂—N—, 2×CH₂—S— overlapped), 2.20-1.92 (24H, 8×OCH₃).

### Synthesis of ethylenedicysteine deoxyglucosamine 3

Fully-protected ethylenedicysteine deoxyglucosamine 7 (1.00 g, 0.73 mmol) was dissolved in ammonia liquid (100 ml) under an argon atmosphere and cleaned sodium metal (0.334 g, 14.5 mmol, 20.0 equivalents) was added in small portions. The reaction mixture turned a deep blue colour and was stirred for 15 min at RT before the addition of small amounts of ammonium phenyl acetate to quench the unreacted sodium metal. The resultant milky white solution was dried under a stream of argon gas to afford a strong-smelling cream-coloured solid. The crude product was handled under an inert atmosphere with the exclusion of light. 2-Propanol (200 ml) was added to the material and stirred vigorously for 10 min before vacuum filtration. The resultant cream-coloured precipitate was washed with diethyl ether and then dried for 2 hours on a high vacuum to afford 0.230 g (53% yield) of the sodium salt of ethylenedicysteine deoxyglucosamine (ECDG) 3. The product was confirmed by ¹H NMR, HPLC and MS analysis which was in accordance with the literature data. C₂₀H₃₈O₁₂N₄S₂ requires 590.665, of which 591.1 was observed.

### REFERENCES

1. http://clinicaltrials.gov/show/NCT01394679, 20 Aug. 2013
2. Zhang, Y. H., Bryant, J., Kong, F. L., Yu, D. F., Mendez, R., Kim, E. E. & Yang, D. J., 2012, Molecular Imaging of Mesothelioma with ^{99m}Tc-ECG and ⁶⁸Ga-ECG, Journal of Biomedicine and Biotechnology, 2012.
3. Zaman, M., 2007, ^{99m}Tc-EC-deoxyglucose—a poor man's ¹⁸F-FDG: what will be the future of PET in molecular imaging?, European Journal of Nuclear Medicine and Molecular Imaging, 34, 427-428.
4. http://www.health24.com/Medical/Cancer/Facts-and-figures/South-Africa-78-increase-in-cancer-by-2030-20120721, 20 Aug. 2013.
5. Yang, D., Kim, C., Schechter, N. R., Azhdarinia, A., Yu, D., Oh, C., Bryant, J. L., Won, J., Kim, E. & Podoloff, D. A., 2003, Imaging with ^{99m}Tc-ECDG targeted at the multifunctional glucose transport system: feasibility study with rodents, Radiology, 226, 465-473.
6. Zhang, Y., Mendez, R., Kong, F., Bryant, J., Yu, D., Kohanim, S., Yang, D. & Kim, E., 2011, Efficient synthesis of ^{99m}Tc-ECDG for evaluation of mesothelioma, Journal of Nuclear Medicine, 52 (Suppl. 1), 1532
7. Ebrahimabadi, H., Lakouraj, M. M., Johari, F., Charkhlooie, G. A., Sadeghzadeh, M., 2006, Synthesis, characterization and biodistribution of ^{99m}Tc-(EC-DG), a potential diagnostic agent for imaging of brain tumors, Iranian Journal of Nuclear Medicine, 14 (Suppl. 1), 36-37
8. Yang, D. J., 2008, US Patent Application US20080107598
9. Blondeau, P., Berse, C., Gravel, D., 1967, Dimerization of an intermediate during the sodium in liquid ammonia reduction of L-thizolidine-4-carboxylic acid, Canadian Journal of Chemistry, 45, 49-52
10. Assad, T., 2011, Synthesis and Characterization on novel benzovesamicol analogues, Turkish Journal of Chemistry, 35, 189-200.
11. Mang'era, K. O & Verbruggen, A., 1999, Synthesis and Evaluation of β-Homocysteine Derivatives of ^{99m}Tc-L,L-EC and ^{99m}Tc-L,L-ECD, Journal of Labelled Compounds and Radiopharmaceuticals, 42, 683-699.

1. A kit for preparing a radioactive labelled ligand, suitable for use as an injectable radiopharmaceutical, the kit comprising:
a) a ligand dissolved in a non-aqueous solvent, the ligand being capable of bonding to a radionuclide and wherein the solvent is selected from any one or more solvents within the relative polarity range of hexane to glycerine;
b) a reducing agent;
c) a buffer solution;
and wherein components a), b) and c) are each in a lyophilized form.

2. The kit according to claim 1, further comprising a component d) comprising additives selected from any one or more of a weak chelating agent, anti-oxidant, solubiliser and a bulking agent, and wherein component d) is in a lyophilized form.

3. The kit according to claim 1, wherein the reducing agent is a mixture of SnCl₂ or SnF₂ or stannous tartrate; hydrochloric acid and water.

4. The kit according to claim 1, wherein the buffer is selected from any one or more of a phosphate, citric acid and acetate buffer solution.

5. The kit according to claim 2, wherein the weak chelating agent is selected from any one or more of DTPA, glucoheptonate, tartrate and medronate.

6. The kit according to claim 2, wherein the anti-oxidant is selected from any one or more of gentisic acid, ascorbic acid and para amino benzoic acid.

7. The kit according to claim 2, wherein the solubiliser is selected from gelatin or cyclodextrin, or a combination thereof.

8. The kit according to claim 2, wherein the bulking agent is selected from any one or more of mannitol, inositol, glucose and lactose.

9. The kit according to claim 2, wherein components a), b), c) and optionally d) are contained in one vial.

10. The kit according to claim 2, wherein components b), c) and optionally d) are contained in a first vial and component a) is contained in a second vial.

11. The kit according to claim 1, wherein the ligand is selected from any one of ECDG, ECD, HMPAO, MAG3, and MIBI; or alkali metal salts, or alkaline earth metals thereof.

12. The kit according to claim 1, wherein the solvent is selected from any one or more of methanol, ethanol, ethyl acetate, hexane, chloroform, dichloromethane, toluene, ether, tetrahydrofuran and acetonitrile.

13. The kit according to claim 12, wherein the solvent is methanol.

14. The kit according to claim 1, wherein the radionuclide is selected from ^{99m}Tc, ¹⁸⁸Re, ¹⁸⁶Re, ¹⁵³Sm, ¹⁶⁶Ho, ⁹⁰Sr, ⁹⁰Y, ⁸⁹Sr, ⁶⁷Ga, ⁶⁸Ga, ¹¹¹In, ¹⁵³Gd, ⁵⁹Fe, ⁵²Fe, ²²⁵Ac, ²¹²Bi, ⁴⁵Ti, ⁶⁰Cu, ⁶¹Cu, ⁶²Cu, ⁶⁴Cu, ⁶⁷Cu, ^{195m}Pt, ^{191m}Pt, ^{193m}Pt, ^{117m}Sn, ¹⁰³Pd, ^{103m}Rh, ⁸⁹Zr, ¹⁷⁷Lu, ¹⁶⁹Er, ⁴⁴Sc, ¹⁵⁵Tb, ¹⁴⁰Nd, ¹⁴⁰Pr, ¹⁹⁸Au, ¹⁰³Ru, ¹³¹Cs, ²²³Ra, ²²⁴Ra and ⁶²Zn.

15. The kit according to claim 14, wherein the radionuclide is ^{99m}Tc, ¹⁰³Pd, ^{103m}Rh, ^{195m}Pt, ^{193m}Pt, ¹⁹¹Pt.

16. The kit according to claim 15, wherein the radionuclide is ^{99m}Tc.

17. The kit according to claim 1, further comprising instructions for use.
